(19) United States
(12) Patent Application Publication
(10) Pub. No.: US 2007/0224599 A1
(43) Pub. Date: Sep. 27, 2007

(54) INTERACTION OF STRESSFUL LIFE EVENTS AND A SEROTONIN TRANSPORTER POLYMORPHISM IN THE PREDICTION OF EPISODES OF MAJOR DEPRESSION

(76) Inventor: Kenneth S. Kendler, Richmond, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/381,039

(22) Filed: May 1, 2006

Related U.S. Application Data
(60) Provisional application No. 60/784,469, filed on Mar. 22, 2006.

(51) Int. Cl. C12Q 1/68

(52) U.S. Cl. 435/6

(57) ABSTRACT

People with the SS variant of the human serotonin transporter gene are more susceptible to episodes of major depression when faced with mildly threatening life events than people with the SL or LL variants. Methods for identifying and treating such patients are provided.

### RELATED APPLICATION

This application claims benefit of U.S. application No. 60/784,469 filed Mar. 22, 2006.

### STATEMENT REGARDING FUNDING

Funding was received from NIMH Grant #40828 and Mental Health, Alcohol and Drug Abuse Grant #49492 from the NIH and AA-09095.

### FIELD OF THE INVENTION

The present invention relates to identifying and treating individuals at risk for developing major depression. In particular, the invention focuses on the SS variant of the human serotonin transporter gene, and identifies and treats people with that gene, such individuals being more susceptible to episodes of major depression when faced with mildly threatening life events than people with the SL or LL variants.

### BACKGROUND OF THE INVENTION

Major depression, which afflicts large numbers of people in the US (and worldwide) is a crippling disorder. It is known to cause general disruption of normal life activities (work, relationships, etc.) and frequently leads to suicide. While serious bouts of depression are often associated with traumatic, stressful life events (SLEs), it is well recognized that not all individuals react to traumatic events in the same way. An event that is incredibly stressful for one individual is not necessarily so for another. Some individuals are stress-sensitive and prone to depression in response to modest stressors while others are stress-resistant, remaining symptom free after severe adversity. It would be beneficial to understand the factors that bring about these differences, and to be able to predict which individuals are likely to succumb to depression as a result of SLEs, in order to presymptomatically intervene with therapies that counter depression.

Clinical depression has been associated with low levels of the neurotransmitter serotonin. Serotonin is characterized as the “feel good” neurotransmitter, and its availability determines a decrease in negative emotions and concomitant increase in positive emotions. A delicate balance exists between serotonin release, or firing, within the synapse and activity of an auto-receptor (the 5HT1A auto-receptor) that inhibits the nerve's firing and the release of serotonin. Both activities are essential. In addition, the serotonin transporter gene (the 5-hydroxytryptophan or “5-HTT” gene) is responsible for making a protein that performs the task of reuptaking, or removing, serotonin at the appropriate time from the site of its activity in the brain-within the synapse, the area of communication between neurons. The transporter protein removes the serotonin at the appropriate time, terminating its effect. It is postulated that removal of serotonin by the transporter protein is necessary for proper neurotransmitter function: without proper removal, the auto-receptor protein causes a net decrease in the overall effect of the serotonin, and lack of availability of serotonin results in an increase in negative emotions.

The 5-HTT gene linked polymorphic region is located in the promoter region of the serotonin transporter gene. In this region an additional 44 base-pairs (alleles) may be present resulting in what is termed the “long” or “1” variant. The same region without the 44 extra base pairs is termed a “short” or “s” variant. The long variant makes twice as much transporter protein as the short variant that is lacking the extra 44 alleles.

Individuals inherit two copies of the serotonin transporter, one copy from each parent. Approximately 17% of the population carries two copies of the short variant, approximately 32% carry two copies of the long variant and approximately 51% carry one copy of each. Those carrying one or two copies of the short variant have a reduced amount of the transporter protein and therefore less efficient processing of serotonin.

Previous work has shown a connection between the 5-HTT genotype of an individual and the individual's likelihood to experience major depression in response to stressful life events. United States patent application 2005/0037405 (Caspi et al., published Feb. 17, 2005, titled “Method for assessing behavorial predisposition”, which is herein incorporated by reference) teaches that persons with an “S” allele (both homozygous SS and heterozygous SL) were significantly more likely to develop depression in response to stressful life events than were individuals with the LL genotype. See also United States patent application 2005/0009035 (Caspi et al., published Jan. 13, 2005, titled “Method for assessing behavorial predisposition”, which is herein incorporated by reference); Caspi et al., “Influence of Life Stress on Depression: Moderation by a Polymorphism in the 5-HTT Gene,” Science, 301:386-389 (2003).

There have been three studies which have examined interactions between 5-HTT genotype, stress and depression. Gillespie et al ³⁴ failed to replicate either a direct effect of the 5-HTT polymorphism on depression or an interaction with SLEs ³⁴. SLEs were assessed over a one-year period using self-report measures so that, like the Caspi et al report, a close temporal resolution for the association between SLEs and depressive onsets was not possible. Eley et al ³⁵ studied self-report depressive symptoms in adolescents and found a trend for an interaction between 5-HTT genotype and a composite measure of environmental risk in the prediction of depression that reached significance in female subjects. Examining a very different outcome, Grabe et al ³⁶ found, in a general adult sample, a significant interaction between unemployment and the 5-HTT genotype in the prediction of chronic disease burden in women but not in men. Further work will be needed to clarify whether and how the 5-HTT gene modulates the pathogenic effects of SLEs and other social stressors.

For other background on serotonin transporter genes, see Kaufman et al., “Social supports and serotonin transporter gene moderate depression in maltreated children,” PNAS 101:17316-17321 (2004); Hariri et al., “A Susceptibility Gene for Affective Disorders and the Response of the Human Amygdala,” Arch Gen Psychiatry 62:146-152 (2005); Pezawas et al., “5-HTTLPR polymorphism impacts human cingulated-amygdala interactions: a genetic susceptibility mechanism for depression,” Nature Neuroscience (8 May 2005).

The following are also cited as background in the patent literature concerning stress and/or depression:

U.S. Pat. No. 6,599,243 (issued Jul. 29, 2003 to Woltermann et al. of DaimlerChrysler AG), for “Personalized driver stress prediction using geographical databases”;

U.S. Pat. No. 6,322,503 (issued Nov. 27, 2001 to Sparhawk, Jr.) for “Method of diagnosing, tracking, and treating depression”;

U.S. Pat. No. 6,317,731 (issued Nov. 13, 2001 to Lulciano) for “Method for predicting the therapeutic outcome of a treatment”;

U.S. Pat. No. 6,063,028 (issued May 16, 2000 to Luciano) for “Automated treatment selection method”;

U.S. Pat. App. No. 20030144829 by Geatz et al. (published Jul. 31, 2003), for “System and method for sensing and evaluating physiological parameters and modeling an adaptable predictive analysis for symptoms management”.

### BRIEF SUMMARY OF THE INVENTION

Humans differ widely in their psychological response to stressful life events. The current ability to predict stress responsiveness in humans is relatively limited. An additional variable not having received particular attention to date is that stressful life events may be of varying levels of seriousness, from mild to serious. The present inventor considered that it would be useful, for example, to know of genetic variant(s), if any, that particularly influence the sensitivity of individuals to the depressing effect of mildly stressful life experiences.

The present invention is based on the discovery that a homozygous short-short (“SS”) 5-HTT genotype predisposes individuals to a significant risk of major depression following mild to moderate stressful life events, compared to heterozygous short-long (“SL”) or homozygous long-long (“LL”) individuals. In other words, SS individuals are more susceptible to the depressogenic effects of even relatively mild or moderately threatening events that occur in their lives, than are persons who possess at least one 5-HTT 1 allele. Based on this discovery, an individual may be screened or clinically assessed and based on the screening results or the clinical assessment may be correspondingly identified as being or not being at risk for developing depressive symptoms. With such screening and identification information, advantageously, therapeutic or psychological intervention may be provided to reduce or prevent the manifestation of depressive symptoms. Additionally, the individual may be informed about life style or career choices that would be beneficial, and information related thereto.

Additionally, the present inventor has now recognized the importance of distinguishing between the relative threat levels of the stress, and is the first to have investigated whether the 5-HTT genotype of an individual influences the response to stress equally for mildly threatening events versus events that were highly threatening. Because humans differ widely in their psychological response to stressful life events, it is particularly significant that the present invention provides identification of a genetic variant that particularly influences the sensitivity of individuals to the depressing effect of mildly stressful life experiences.

Advantageously, the present invention (especially when used, e.g., with a kit comprising a genetic test or a kit comprising a genetic test and a scale) facilitates the counseling of an individual on the basis of the individual's genotype about the individual's possible stress responsivity (such as, e.g., the individual's stress responsivity to mildly threatening events, etc.). Because an individual without such assessment, screening and counseling would be unlikely to otherwise expect mildly threatening events to necessarily affect him or her, useful information and assistance may thereby be provided to at risk individuals. Such information could be particularly helpful, for example, during convalescence from a major health trauma. The deleterious effect of depression on the ability of patients to recover from medical trauma is well known. Without the information provided by the invention, health care providers could be “caught off-guard” when, for example, a patient who is expected to do well, instead succumbs to depression and consequently does not recover as predicted. In other words, although the patient may be expected to handle the stress of the major medical trauma (e.g. heart attack, surgery, etc.) in a manner that is deemed normal or typical, the major medical trauma may be coupled with other relatively mild stressful life events that may subsequently put an SS individual at risk for major depression, which in turn impairs his or her ability to recover from the trauma. Without the methods of the present invention, such a predisposition would not be factored into the overall treatment plan of the patient and would not allow the treating physician to take medical and psychological measures to better protect his at risk patient.

Additionally, advantageously the individual with the SS allele may benefit from counseling and information that may be provided for him for educational choices, career planning, financial planning, planning for medical insurance or family planning. The SS individual may, by better understanding his or her SS status, make choices that for him/her are of significant benefit and to the benefit of employers, insurers, family and personal satisfaction and feelings of accomplishment.

Advantageously, also, the person who has suffered from major depression but who has not experienced major stressful life events, but rather only mildly stressful life events, may not be forewarned or consider that he may be suffering from major depression. He may be conditioned to believe that something extremely “bad” must have happened to him for him to be depressed, and may not seek medical or psychological assistance. The individual may turn instead to self medication (e.g. drugs, alcohol, etc.) with disastrous results, and/or develop feelings of self-loathing, anger, and frustration, which may manifest as exhibitions of anger and violence. An SS individual may fall victim to this behavior without knowing that mildly stressful life events, for him, must be factored in and that he may be suffering form major depression.

The invention provides a method of identifying an individual that is at risk for an episode of major depression. The method includes the steps of 1) identifying an individual that possesses an SS allelic variant of human serotonin transporter gene; 2) providing a scale which correlates life events experienced by human beings with a level of severity, wherein at least some of said life events on the scale are deemed to be of mild or low moderate long term contextual threat; 3) determining whether the individual has or will experience a life event which has a severity of mild or greater on the scale; and, if the individual has or will experience the life event, 4) concluding that the individual is at risk for an episode of major depression. In one embodiment, the life event has a severity of mild or low moderate on the scale.

The invention further provides a method for treating an individual that is either at risk or is experiencing an episode of major depression. The method comprises the steps of 1) identifying an individual that possesses an SS allelic variant of human serotonin transporter gene; 2) providing a scale which correlates life events experienced by human beings with a level of severity, wherein at least some of the life events on the scale are deemed to be mild or low moderate long term contextual threat; and 3) determining whether the individual has or will experience a life event which has a severity of mild or greater on the scale; and, if the individual has or will experience the life event, 4) providing the individual with a treatment which alleviates or prevents an episode of major depression. In one embodiment of the invention, treatment includes counseling. In another embodiment, treatment includes pharmaceuticals. The method may include presymptomatic identification of an at risk individual.

The invention further provides a preventative medicine kit, comprising: (a) a genetic test administrable to a human individual for determination of whether the individual possesses at least one genetic polymorphism; and (b) a scale which correlates life events experienced by human beings with a level of severity, wherein at least some of the life events on the scale are deemed to be mild or low moderate long term contextual threat. In one embodiment of the kit, the genetic test when administered to the individual provides a result for whether the individual possesses an SS allelic variant of human serotonin transporter gene.

The invention also provides another method of identifying an individual that is at risk for an episode of major depression. The method comprising the steps of 1) identifying an individual that possesses an SS allelic variant of human serotonin transporter gene; 2) determining whether the individual has or will experience a stressful life event; 3) assessing whether the stressful life event is of mild or low moderate long term contextual threat; and, if the individual has or will experience the life event, 4) concluding that said individual is at risk for an episode of major depression.

The invention further provides an additional method for treating an individual that is either at risk or is experiencing an episode of major depression. The method comprises the steps of 1) identifying an individual that possesses an SS allelic variant of human serotonin transporter gene; 2) determining whether the individual has or will experience a stressful life event; 3) assessing whether said stressful life event is of mild or low moderate long term contextual threat; and, if said individual has or will experience said life event, 4) concluding that the individual is at risk for an episode of major depression; and, if the individual has or will experience said life event, 5) providing the individual with a treatment which alleviates or prevents an episode of major depression. Treatment may include, for example, counseling and/or pharmaceuticals. The method may include presymptomatic identification of an at risk individual.

### BRIEF DESCRIPTION OF THE DRAWINGS

FIG. 1. The hazard ratio of onset of major depression within a 2 month period as a result of i) sex (men versus women), ii) genotype at the 5-HTT polymorphism (SS versus LS/LL), iii) the occurrence, in the first month, of a stressful life event. A hazard rate of unity was defined as the risk level for a male with an SS genotype and no life-event exposure.

FIGS. 2A and 2B. The hazard ratio of onset of major depression within a 2 month period as a result of i) sex (men versus women), ii) genotype at the 5-HTT polymorphism (SS versus LS/LL), iii) the level of long-term contextual threat experienced in the first month, broken down into 4 levels: i) no threat (no stressful life event [SLE] exposure); ii) exposure to a SLE rated as having minor or low-moderate threat; iii) exposure to a SLE rated as having high-moderate threat and iv) exposure to a SLE rated as having severe long-term contextual threat. A hazard rate of unity was defined as the risk level for a male with an SS genotype and no life-event exposure.

FIG. 2A presents the full results, while FIG. 2B “zooms in” on the crucial part of the curve reflecting changes in response to minor and low-moderate threat.

### DETAILED DESCRIPTION OF THE INVENTION

The present invention is based on the discovery that individuals with the SS 5-HTT genotype are at greater risk for developing major depression in response to mild or moderately threatening stressful life events, than are individuals with the SL or LL 5-HTT genotype. This discovery allows the identification of such individuals, and the development of interventional therapies to lessen or prevent depressive symptoms in them.

Such individuals may include, for example, “patients” in the usual sense of the word, e.g. persons who are or should be under the care of a health care professional. Alternatively, such individuals may be persons who are assessed by the methods of the invention for other reasons that are not strictly “clinical” in the usual sense of the word. Examples include but are not limited to clients who are working with a life-style coach, a fitness instructor, or others whose job typically involves guiding clients toward more healthy lifestyles, toward extending longevity, increasing productivity, enhancing relationships, assessing aptitude for various careers, making decisions regarding health and life insurance, etc. In these cases, the individual is usually involved in and aware of the assessment procedure, and the assessment is intended to benefit the individual in some way, particularly with respect to future life-style planning. In some cases, the individual may be a child or dependent (or even a fetus) whose caretakers (e.g. parents, educators, guardians, etc.) may wish to optimize the environment of the child or dependent to enhance the chance of future success in education, formation of relationships, job and career performance, and other undertakings. Other scenarios for use of the invention include screening procedures that may or may not be ultimately used to benefit the individual, but rather to benefit an interested party such as a potential employer, an insurance provider, government agency, etc. In these cases, the interested party may use the methods, for example, as a means to “weed out” or reject the applications of individuals who may be susceptible to major depression as a result of mildly stressful life events. In the case of health, disability or life insurance providers, the information may be used to reject applicants, or to increase the premium that is charged for insurance.

“Major depression” herein means that an individual exhibits symptoms, as evidenced, for example, by assessment methods that are known in the art (e.g. Diagnostic Interview Schedule, etc.) consistent with art recognized diagnosis of depression according to DSM-V criteria. Essentially, a major depressive episode is a period of at least two weeks during which there is either depressed mood or the loss of interest or pleasure in all activities. To be considered to suffer from “major depression,” an individual must also experience four of the following additional symptoms: changes in weight or appetite, sleep, or psychomotor activity; decreased energy; feelings of worthlessness or guilt; difficulty thinking or concentrating; or recurrent thoughts of death or suicidal ideation. Further, the episode must be accompanied by clinically significant distress or impairment in social, occupational or other important areas of functioning.

A scale has been mentioned for use in the inventive methods, such as, e.g., a scale ranking stressful life events based on the threat level represented, e.g. from low threat level or mild threat level to very high threat level or extremely threatening. Life events that are so ranked may include those that are recognized by those of skill in the art as usually impacting an individual's sense of well being, and include but are not limited to an adverse health effect, assault, legal problems, financial setback, loss of a relationship, loss of a loved one, job loss, interruption of a relationship and/or activity that was previously relied upon for a sense of well being, illness in a loved one, serious interpersonal conflict, etc. According to the invention, such events are ranked according to the perceived threat level that the event poses to the individual who is being assessed by the inventive methods, and a scale of the ranking is provided and used, in conjunction with determining the patient's 5-HTT genotype, to assess a patient's vulnerability to major depression. Such a scale may be provided in many forms known to those of skill in the art, for example, as a list or diagram with groups of events classified under headings such as, e.g. mild, moderate, severe, etc. Those of skill in the art will recognize that the perception of the threat level of an event (i.e. whether an event represents a low, moderate or high threat level) will vary greatly from one individual to another, and among social groups. Perception by an individual depends on numerous factors, such as cultural norms and practices, age at the time of the event, resources available to cope with the event, previous experience dealing with challenges, religions beliefs, etc. A scale used in the practice of the invention is formulated in a manner recognized by professionals practicing in a relevant field (e.g. psychiatry, psychology, social work, etc.) and is contextual in nature, i.e. varies depending on the social/cultural context of the individuals being assessed. For example, a structured interview and associated training manual with examples may be prepared and used that allows individuals with basic mental health, educational, or human resources training to reliably rate the threat level for experienced stressful events. For the present study, a scale for measurement of long term contextual threat first developed by George Brown (G. W. Brown and T. O. Harris. Social Origins of Depression: A Study of Psychiatric Disorder in Women, London: Tavistock, 1978) was adapted and modified. Additional resources for rating event severity include, for example: G. W. Brown and T. O. Harris. Life Events and Illness, New York: Guilford Press, 1989.

In other embodiments of the invention, a scale per se is not utilized. Rather, a person with an interest in the individual being assessed (or the individual themselves) makes the judgment that the SLE is of a mild, minor or moderate threat level. Such persons would typically be, for example, health care or other professionals (for example, a physician, a life-style coach, employer, etc.) who would be aware of the usual perception of the SLE in the environment of the individual, and would therefore be capable of making such a judgment call. Those of skill in the art will recognize that, in many circumstances, the use of a formal scale or listing of mildly threatening SLEs would not be necessary in order to provide a substantial benefit to an individual with an SS genotype. In many situations, the affected individuals themselves are well-qualified to make such an assessment, i.e. to determine whether an impending SLE is likely to be mildly stressful, or whether a particular event has been mildly stressful.

In general, stressful life events with mild or low moderate long-term contextual threat are characterized by one or both of two key features. First, there is a relatively rapid resolution of the problems posed by the event, typically within a 2 week period. Second, the events, while they may involve inconvenience or embarrassment, pose no significant threat to an individual's key roles or self concepts. In one embodiment of the invention, examples of the stressful life event that is of a long-term contextual threat level that is mild, minor or only moderately elevated, include the following:

1. Minor threats are events presenting little or no threat or events where all of the negative implications are cleared up within 10-14 days. Examples (where R is the individual experiencing the stressful event) include but are not limited to:

R is told his child may have diabetes but within a week this is revised to a less serious condition. Child will not require regular medical care.

R's 80 year-old mother fell and broke hip. Hospitalized and recuperated for a month. R helped brother care for mother during that time. R wasn't working at the time so able to give the time. Interfered very little with social activities. No financial effects. R was happy to help out.

Wife broke arm in three places slipping on wet floor at the supermarket. She is out of cast and moving arm ok but still has some pain. R had to take over some household responsibilities while it was healing. Minor inconvenience and a little worry about her recovery.

R's grandmother died. Expected. Long declining health. Not very close to her.

R ended a romantic relationship. Not unexpected. A realization of differences in interests. He was older and didn't want children. He's still R's best friend and there's regular contact.

Landlord threatens to evict R but within a few days changes his mind and gives R new lease. R had begun to move belongings into storage.

R is threatened with a lawsuit but this is dropped within a week.

R is fired from a part-time job but had been planning to quit anyway.

Rumor went through office that R's work section would be laid off in 1 week. R extremely worried and anxious but rumor turned out not to be true.

R bought new living room, kitchen, and bedroom furniture. R used some of savings and charged the rest. He is due an inheritance and will pay off the charge card bill using some of that money. He prefers to charge purchases even though he has the cash to pay off because he likes to earn frequent flier miles.

R not doing well in 2 college classes—got 2 D's. Knew he still had enough credits to graduate and planned to retake the classes in summer to raise his grades.

2. Low moderate threats have significant negative implications but are not likely to cause most people in R's position to feel a great deal of severe long-term threat. These include long-term worries which are not likely to erupt immediately into a severe problem.

R diagnosed with diabetes. Doctor said is mild form but R must stay on a special diet the rest of life. R will require regular medical monitoring but otherwise can lead normal life.

Daughter was suspended from school for 10 days for fighting. R put daughter on house restriction for that time and daughter argued and complained to R all the time. Relationship returned to normal after she returned to school.

R and husband separated for a couple of weeks. Though no serious difficulties getting along, felt they had been growing apart. They've talked about separating off and on over the past few months so this was not unexpected. R and husband talked a lot during the time apart and R is confidant they have grown closer as a result.

R and mother had bitter argument about the way R was “neglecting” her children. Have always had conflict over this but this argument was worse and led to them not speaking to each other for a week or two. R and mother are not close.

R returns to work after medical leave and is unsure she will be able to handle her job as well as before. After about three weeks R feels fully back in the swing of things.

R laid off due to defense cutbacks. R knew it was coming and company gave job counseling and 1 month severance pay. R found another job within a couple of weeks and enjoyed the time off.

Boss didn't do his share of work on a big project. R had to work longer hours to get the project done and grew to resent boss. They got along before this but since things have been tense. R now doesn't put as much effort into work as he used to.

R went on extended maternity leave after birth of son. Not unexpected. Financial situation has been tight for past 3 months because additional costs of baby.

House infested with roaches. R had to stay in motel for 2 weeks during extermination. House was “tented”. House almost condemned and R worried he would lose investment and home. R thinks he could have called exterminator sooner.

R's brother lost job. Was unexpected. R consoled and was more attentive to him for a month or so. R also helped him out financially for a few months until he found new work. Some financial impact on R. R says he and brother became closer.

House robbed while R on vacation. Insurance covered most costs to replace what was stolen. R had security system installed but still worries a lot about it happening again.

When an individual is identified as an SS genotype individual, or when an individual is identified as an SS genotype individual and a scale according to the invention is used, therapeutic intervention may be concluded to be useful. Therapeutic intervention for SS individuals may take many forms. Such intervention may be prophylactic and undertaken prior to the onset of depression. Intervention may be undertaken when it is known that a potentially stressful event is likely or certain to occur, for example, a necessary medical procedure, imminent loss of a loved one, etc. Intervention may include any therapeutic or educational methods known to those of skill in the art which are efficacious in decreasing or dealing with stress and/or negative feelings that may be induced by stress. Examples include but are not limited to: various medications; counseling; the development of both professional and non-profession support systems; biofeedback and other relaxations techniques (exercise, meditation, etc.); acquisition of a pet; removal of additional potential sources of stress, etc.

Alternatively, even if no such potentially stressful event is foreseeable, an SS individual may benefit from understanding that he or she is at risk, and taking measures in advance to decrease their exposure to stressful events, and to prepare for the possibility of their occurrence. For example, an SS individual may be encouraged to cultivate and carefully monitor his or her coping mechanisms, establish ongoing relationships with mental and other health care professionals, increase non-professional social support networks, improve general physical health, deal with potential sources of stress before they “get out of hand” (e.g. to budget time, finances and other resources realistically), etc. Also, for some individuals, medications which elevate and/or stabilize mood may be beneficial.

Methods of establishing the genotype of an individual are well-known to those of skill in the art. The particular methods used in the development of the present invention are described in the Examples section below. However, other methods are known and may be utilized, so long as the protocols involved specifically and accurately measures the sizes of the nucleic acid fragments of interest (in this case, of the long and short 5-HTT alleles) and allows the investigator to distinguish between the three possible genotypes (SS, SL and LL).

The methods of the present invention may be used alone in order to address an individual's predisposition to depression as a result of mild to moderate threat-level stress. Alternatively, the invention also contemplates the inclusion of the detection of the 5-HTT genotype with the detection of other genetic profiles of interest. Such genetic profiles may also be related to the predisposition to depression or other mental disorders, and examples include but are not limited to the risk for disorders that may be etiologically related to depression such as anxiety and personality disorders, learning disorders, medical disorders such as Parkinson's Disease, multiple sclerosis, Chronic Fatigue Syndrome, HIV disease, chronically elevated cortisol disorders, infectious diseases such as Lyme disease, or Epstein-Barr disease, etc.

The invention may be further appreciated from the following Examples, without the scope of the invention being limited thereto.

### EXAMPLES

### Example 1

Prior evidence from twin studies suggested genetic moderation of the depressogenic effects of stressful life events (SLEs). The question under consideration was whether the specific genes involved in this effect can be identified. An objective of this research was to replicate and extend a recent study that a functional variant in the serotonin transporter (5-HTT) might in part explain these findings. This was carried out by characterizing risk for major depression and generalized anxiety syndrome in the last year as a function of 5-HTT genotype, sex and the occurrence of SLEs and ratings of the SLE-associated level of threat in a population-based sample of adult twins. Five hundred and forty-nine male and female twins with a mean age at participation of 34.9 years (SD=9.1) participated in the study. The main outcome measure was episodes of major depression and generalized anxiety syndrome in the last year with onset measured to the nearest month.

The results showed that individuals with two short-alleles at the 5-HTT locus were more sensitive to the depressogenic effects of all SLEs than were those with one or two long alleles. When the level of SLE-associated threat was examined, the interaction between genotype and SLE resulted from an increased sensitivity of SS individuals to the depressogenic effects of common low-threat events. These events had little impact on risk for those possessing the SL and LL genotypes. The 5-HTT genotype did not modify the effects of SLEs on risk for generalized anxiety syndrome. These results showed that variation at the 5-HTT moderates the sensitivity of individuals to the depressogenic effects of SLEs largely by producing, in SS individuals, an increased sensitivity to the effects of mild stressors. Replication of these intriguing results is needed.

Stressful life events (SLEs) precede the onset of episodes of major depression (MD) more frequently than expected by chance ^{1 2} and this relationship is probably causal ³. However, humans display wide variation in response to adversity. Some individuals are stress-sensitive and prone to depression in response to modest stressors while others are stress-resistant, remaining symptom free after severe adversity ⁴. The question in this Example is the source of this variation.

It was previously demonstrated in an adult twin sample that genes, assessed in aggregate, impacted on sensitivity to the depressogenic effects of SLEs ⁵. Similar results have been found in adolescent twins ^{6 7}. However, these studies did not examine the specific genes involved in this effect.

Recently, in a New Zealand birth cohort, Caspi and coworkers reported that a functional length polymorphism in the promoter of the serotonin transporter (5-HTT) gene moderated the influence of SLEs on depressive symptoms and MD ⁸. They found that individuals with one or two “short” alleles at this polymorphism (hereafter SL and SS, respectively) were more stress-sensitive than those with two “long” alleles (hereafter LL). Their analyses had three potential methodologic limitations. First, they predicted past-year MD assessed at age 26 from the sum of 14 possible SLEs in the preceding 5 years. However, the impact of SLEs on risk for MD is typically short-lived, usually one to three months ^{9 10 11 12 13}. Their results may reflect, at least in part, an indirect rather than a direct association between SLEs and MD. Second, the pathogenic effects of SLEs are highly variable 14 and related to their associated level of threat ^{15 16 13} Examining the SLE-associated threat level along with the 5-HTT genotype may better characterize the nature of genetic effects on stress-responsivity. Third, their study did not address the specificity of the 5-HTT effect. When assessed at the aggregate level, the genetic risk factors for MD and generalized anxiety disorder (GAD) are closely inter-related ^{17 18}. Given that SLEs also impact on risk for GAD-like syndromes ^{19 13}, the questions was whether the 5-HTT polymorphism would also modify the anxiogenic effects of SLEs.

This Example sets about to replicate the findings of Caspi et al in a random sample of twins from a population-based registry. SLEs and depressive onsets were measured to the nearest month and, for certain interviews, SLEs were rated on a 4 point scale of long-term contextual threat (LTCT). Three questions are addressed: 1) whether the findings of Caspi et al that 5-HTT promoter variation modifies the depressogenic effects of SLEs when the temporal proximity of the SLE to the depressive episode is assured could be replicated; 2) how variation at the 5-HTT polymorphism alters the dose-response relationship between severity of stress and risk for MD; and 3) whether the 5-HTT polymorphism modifies the anxiogenic effects of SLEs.

Methods

Sample

Subjects came from the Virginia Adult Twin Study of Psychiatric and Substance Use Disorders, a longitudinal study of twins drawn from the population-based Virginia Twin Registry²⁰. For female-female (FF) twin pairs, entry criteria required that they be born 1934-1974 and both members had previously responded to a mailed questionnaire in 1987-1988. These FF pairs have been approached for 4 subsequent waves of personal interviews from 1988 to 1997, with cooperation rates ranging from 85 to 92%, here called, respectively, FF1, FF2, FF3 and FF4. For male-male/male-female (MMMF) twin pairs, they were eligible if they were born 1940-1974 and had participated in a first wave interview—termed MMMF1: cooperation rate of 72.4% completed 1993-1996. They were later approached for a second interview—MMMF2, completed 1994-1998—which achieved an 82.6% cooperation rate. For the subsample used in this Example, the relevant inter-wave intervals (±SD) were: FF3-FF4 29.1±5.8 and MMMF1-MMMF2 18.5±7.6 months. After an explanation of the research protocol, informed consent was obtained prior to all interviews.

First, 572 participants were randomly selected from a twin sample who had participated in the MMMF2 or FF4 interviews and had available DNA. Of these twins, 549 had complete data and were included in these analyses. The only selection rules were to never take both members of a twin pair and to select equal numbers of males and females. For every monozygotic twin in the sample with complete data on the cotwin (n=159), phenotypic data were included from the cotwin assuming both twins had identical genotypes. The mean age and years of education of this sub-sample as of May 1996 was 34.9 (SD=9.1) and 13.4 years (SD=2.4), respectively.

Measures

During each interview, the occurrence was assessed over the last year of 14 symptoms representing the disaggregated nine “A Criteria” for MD in DSM-III-R ²¹ (e.g. two items for assessing, separately, insomnia and hypersomnia). For each reported symptom, interviewers probed to insure that it was due neither to physical illness nor medication. The respondents then aggregated these symptoms into co-occurring syndromes the dates of the onset and offset of which were recorded. The diagnosis of MD was made by computer algorithm incorporating the DSM-III-R criteria, except criterion B2 (excluding “uncomplicated bereavement”). In 375 twins interviewed twice by different interviewers with a mean (SD) inter-interview interval of 30 (9) days, the inter-interview reliability of the diagnosis of MD in the last year was: κ²²=+0.66 (95% CIs 0.58-0.74), tetrachoric correlation=+0.88 (0.82-0.93).

In addition, inquiries were made about times in the last year when subjects felt “anxious, nervous or worried,” their “muscles felt tense” or they “felt jumpy or shaky inside.” Positive responses to these probes were followed by questions for all the individual symptoms of DSM-III-R GAD. There was defined a disorder termed “Generalized Anxiety Syndrome” (GAS) lasting 2 or more weeks with a minimum of 6 D criteria for GAD in DSM-II-R ^{21 23}. This definition was used to focus on symptomatic differences between GAS and MD, rather than differences in duration. No diagnostic hierarchy was used between GAS and MD.

Interviews assessed the occurrence, to the nearest month, of 11 personal stressful life events: “assault”, “divorce/separation”, “major financial problem,” “serious housing problems,” “serious illness or injury,” “job loss”, “legal problems”, “loss of confidant”, “serious marital problems,” “robbed” and “serious difficulties at work”. Four classes of network events impacting on spouse, child, parent, sibling, other close relative or “someone else close to you” were assessed. These classes were: i) “getting along with”—serious trouble getting along with an individual in the network, ii) “crisis”—a serious personal crisis of someone in the network, iii) “death”—death of an individual in the network and iv) “illness”—serious illness of someone in the network.

Each SLE in the FF3, FF4 and MMMF2 interviews was rated by the interviewer on the level of long-term contextual threat (LTCT), where “long-term” means persisting at least 10-14 days. Following Brown, interviewers were instructed to rate what most people would be expected to feel about an event in a particular set of circumstances and biography, taking no account either of what the respondent says about his or her reaction or about any psychiatric or physical symptoms that followed it. ²⁴

LTCT was rated on a 4 point scale: minor, low moderate, high moderate and severe Reliability of LTCT ratings was determined by inter-rater and test-retest designs. Inter-rater reliability was assessed by having experienced interviewers review tape recordings of the interview sections in which 92 randomly selected individual SLEs were evaluated. Inter-rater reliability was rₛ=+0.69 and κ=+0.67. Test-retest reliability was obtained by repeating the interview with 191 respondents at a mean interval of 4 weeks. 173 scored life events were obtained that were reported to have occurred within one month of one another and were assumed to represent the same event. Reliability was assessed by Spearman correlation (rₛ) and weighted kappa (κ) ²⁵. The test-retest reliability for LTCT was rₛ=+0.60 and κ=+0.41.

For this study, two different data sets were used. First, only the presence/absence of SLEs was examined in each month using FF3, FF4, MMMF1 and MMMF2 waves. (The FF1 and FF2 waves were not used because of differences in the ways in which SLEs were coded.) This sample contained two strata, the first made up of the FF3 and MMMF1 samples and containing 662 observations or “periods of wellness” of which 46 ended in a depressive episode. A “period of wellness” is defined as a period of observation that either begins at the start of a one-year prevalence window or at the time of recovery from an episode and ends either at the conclusion of that one-year window or at the time of an onset of an episode. The second strata consisted of the FF4 and the MMMF2 samples containing 710 periods of wellness, 44 of which end in an episode of MD.

For the second series of analyses, the LTCT ratings were used and were therefore restricted to the use of the FF3, FF4 and MMMF2 waves. In these analyses, the first strata was represented by the FF3, and MMMF2 waves which contained 662 periods of wellness, 44 of which ended in episodes of MD. Strata 2 was represented solely by the FF4 wave, which contained 299 periods of wellness of which 20 ended in a depressive episode. For GAS only one strata was necessary because coded data on onsets was not available from the FF4 interview, consisting of 662 periods of wellness of which 53 resulted in an episode of GAS.

Genotyping

Cytology brushes were used to obtain a sample of buccal cells from the subjects for DNA analysis. Genomic DNA was isolated using the Instagene Matrix (Biorad) kit protocol for cell lysis product absorption. Each sample was diluted to a working concentration of 5-20 ng/μl. Primer sequences described previously ²⁶, HTTLPR-F (5′-tgaatgccagcacctaaccc-3′) (SEQ ID NO: 1) and HTTLPR-R (5′-ttctggtgccacctagacgc-3′) (SEQ ID NO: 2) were used. PCR products were amplified in 96 well microtitre plates in 20 μl volume containing 50-200 ng human genomic DNA, 0.5 μM each forward and reverse primer, 0.3 mM each dATP, dCTP and dTTP, 0.15 mM dGTP, 0.15 mM 7 deaza dGTP (Amersham, Piscataway, N.J.), 0.4 units Hotmaster Taq (Eppendorf, Westbury, N.Y.), 1× Hotmaster buffer (Eppendorf, Westbury, N.Y.), 1.5 mM MgCl₂, and 0.25 mM. PCR was carried out in a PTC 225 DNA Engine (MJ Research, Waltham, Mass.). Cycling conditions were 5 minutes initial denaturation at 95° C. followed by 40 cycles of 30 seconds at 95° C., 30 seconds at 58° C. and 30 seconds at 72° C., with a final extension of 10 minutes at 72° C. HTTLPR long allele (insertion) of 528 bp and short allele (deletion) of 484 bp were resolved on 2% agarose gels and visualized with ethidium bromide. The frequencies of the 3 genotypes in the sample, which was entirely Caucasian, were: SS—23.3%, SL—47.5% and LL—29.2%.

Statistical Methods

The unit of analysis was a “period of wellness”. Using these periods, analyses were conducted with a Cox proportional hazards model operationalized in the SAS procedure PHREG ^{27 28}. Three predictor variables were used: 5-HTT genotype (LL, SL or SS), sex and either the presence or absence of a SLE or the level of LTCT. When multiple events occurred in the same month, LTCT was coded as the highest recorded threat level. The two dependent variables were: onset of an episode of MD and onset of an episode of GAS.

For analyses incorporating threat levels, LTCT was coded so that 0 meant no SLE occurrence in the month and 1 through 4 meant the occurrence of a SLE with minor, low moderate, high moderate and severe LTCT. To incorporate the ordinal structure and to simplify interpretation of the interaction, LTCT was coded as follows: Four dummy variables X1, X2, X3 and X4 were used. If there was no life event, all four were coded as zero. If there was a significant life event with LTCT of 1 or more, X1 was coded as 1. If LTCT was 2 or more, X2 was also coded as one. If LTCT was 3 or more, then X3 was coded as one. For an event with LTCT=4, all four dummy variables were coded to one. Thus, the coding for a month with an event with a LTCT=2 was: X1=1, X2=1, X3=0, X4=0. This method of dummy variable coding is often referred to as thermometer coding ²⁹. Finally, they were incorporated as a time dependent covariate with a linear decay which abated after two months.

The 5-HTT genotype was coded so that 0 meant two long alleles, 1 meant one long and one short allele and 2 meant two short alleles. To incorporate this into the model, two thermometer type dummy variables, H1 and H2 were used. If there were no short alleles, H1 and H2 were both coded as 0. If there was one short and one long allele, H1 was coded as 1 and h2 was coded as 0. Only when both alleles were short was H2 coded as 1. This allowed easy comparison of two to one short alleles and one short allele to none.

Thermometer coding does not alter model results and is simpler yet mathematically equivalent to contrasts. Compared to typical indicator variables, it greatly simplifies the model selection process. Removal of a level of a variable with standard indicator variables requires a recoding of the data and a likelihood ratio test. With thermometer coding, the same task is no different than removing other independent variables.

The model was produced using two strata to accommodate the first wellness period of two different 13 month time periods for the subjects. At most one onset of MD was used for each time period. If two or more onsets did occur for a subject in the same time period only the first was analyzed. This stratification is a conservative way to deal with in-subject correlation.

Model selection began with the 5HTT genotype, LTCT, sex and all two-way interactions. The final model, which consisted of only significant interactions and main effects which were either significant or were a part of a significant interaction, was obtained by removing non-significant interactions and main effects from the full model. To verify the final model, a random selection of non-significant interactions and main effects was added to the final model to verify that the same model emerged.

The same methods were used to pursue models where LTCT ratings were not available. In these situations, all SLEs were set as though they had an LTCT level of 1 so that the analytic model was constructed similarly for data which included long term contextual threat and data which did not.

The obtained data were examined for whether 5-HTT genotype (along with sex as a covariate) predicted the occurrence of one or more SLEs. The effect did not approach significance (H1 χ₁^{2=1.64}, P=0.20 and H2 χ₁²=0.20, P=0.65). These analyses were then repeated for SLEs with levels of LTCT of 2 or more, 3 or more, and 4 or more. In none of these analyses were the results significant.

Results

Interaction between Event Occurrence and 5-HTT Genotype in the Prediction of Major

Depression

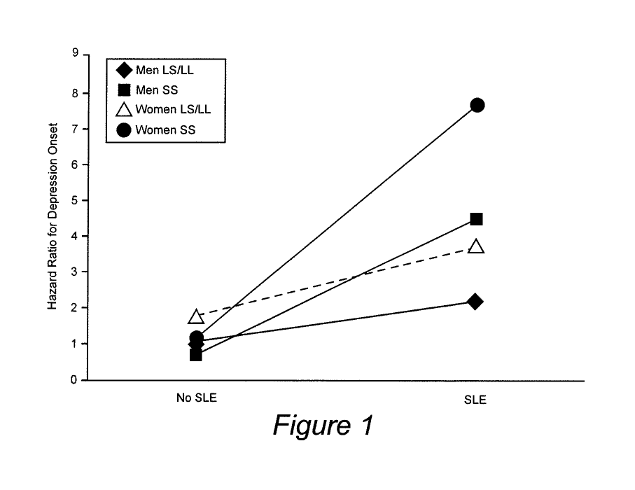

Initial analyses, which included only the presence or absence of SLEs, began with a full model containing the SS, SL and LL genotypes, sex, and the occurrence of a SLE. The model was simplified by combining the effects of SL and LL genotypes with an improvement in fit. This best-fit model, the results of which are shown with CIs in Table 1 and illustrated in FIG. 1, found, for the prediction of episodes of MD, significant main effects for sex (χ₁², 19, p=0.01) and SLE occurrence (χ₁²=7.36, p=0.02) but not for genotype (χ₁²=1.15, NS). However, a significant genotype X SLE interaction was seen (χ₁²=4.34, p=0.04). Estimates based on this model indicate that, averaged across sexes, event exposure increased the hazard ration (HR) for MD in individuals an SL/LL and SS genotype, respectively, 2.13 fold and 6.68 fold.

TABLE 1
Hazard Ratios (and 95% confidence intervals) for Major Depression
as a Function of Stressful Life Event or Threat Exposure, Sex and
5-HTT Genotype as Predicted by Best Fit Statistical Model.

| | Male | | Female | |
|---|---|---|---|---|
| Genotype | LS/LL | SS | LS/LL | SS |
| No SLE | 1 | 0.7 | 1.7 | 1.2 |
| | (reference) | (0.1-1.1) | (1.0-3.8) | (0.2-2.9) |
| Any SLE | 2.1 | 4.4 | 3.7 | 7.7 |
| | (1.3-4.6) | (2.8-12.2) | (1.7-13.2) | (4.0-33.9) |
| No Threat | 1 | 0.5 | 1.4 | 0.6 |
| | (reference) | (0.1-1.1) | (0.7-2.8) | (0.1-1.9) |
| Minor/Low-Moderate Threat | 0.2 | 4.0 | 0.2 | 5.5 |
| | (0.0-0.5) | (1.0-8.9) | (0-0.9) | (1.1-18.0) |
| High Moderate Threat | 4.2 | 5.1 | 5.7 | 7.0 |
| | (1.5-8.7) | (1.1-12.7) | (1.8-15.2)) | (1.8-21.0) |
| Severe Threat | 29.1 | 35.7 | 40.0 | 49.0 |
| | (12.0-64.5) | (8.3-104.1) | (14.4-110.6) | (10.5-170.6) |

Interaction Between Long-Term Contextual Threat Ratings and 5-HTT Genotype in Prediction of Major Depression

Given evidence for an interaction between 5HTT genotype and event exposure in the prediction of MD, how this polymorphism altered the dose-response relationship between severity of stress and risk for depressive onset was explored in this Example.

For the sample containing LTCT ratings, the model could again be simplified by collapsing the genotypic data into the two classes of SS vs. SL/LL. No significant difference was found between the effects of LTCT levels 1 and 2. Sex was retained in the model although its effect fell short of significance. Of the three possible interactions with genotype and LTCT level, two were retained. The final model for the prediction of MD then, contained sex, the main effects of 5-HTT genotype (coded as SS versus SL/LL), the main effects of three levels of LTCT (coded as LTCT levels.≧1, ≧3, or =4) and the interactions between genotype and LTCT value. 1 or more and LTCT.3 or more.

The main effects of 5-HTT genotype (χ₁²=^{2.04}, NS) and LTCT. 1 (χ₁²=3.31, NS) were nonsignificant in this final model. By contrast, the main effects of both levels of stress remained significant: LTCT. 3 or more (χ₁²=9.89, p=0.002) and LTCT=4 (χ₁²=18.66, p<0.0001). Most importantly, in this Example a significant positive interaction was observed between genotype and LTCT. 1 (χ₁²=10.74, p=0.001) such that individuals with the SS genotype had greater sensitivity to the depressogenic effects of SLEs with LTCT levels of mild or greater than did individuals with the SL or LL genotypes. Furthermore, there was a significant, negative and nearly balancing interaction between genotype and LTCT. 3 (χ₁²=6.47, p=0.001). That is, high levels of LTCT were associated with a large increase in risk for MD in all genotypes, whereas low levels of LTCT was associated with increased risk only among individuals with the SS genotype.

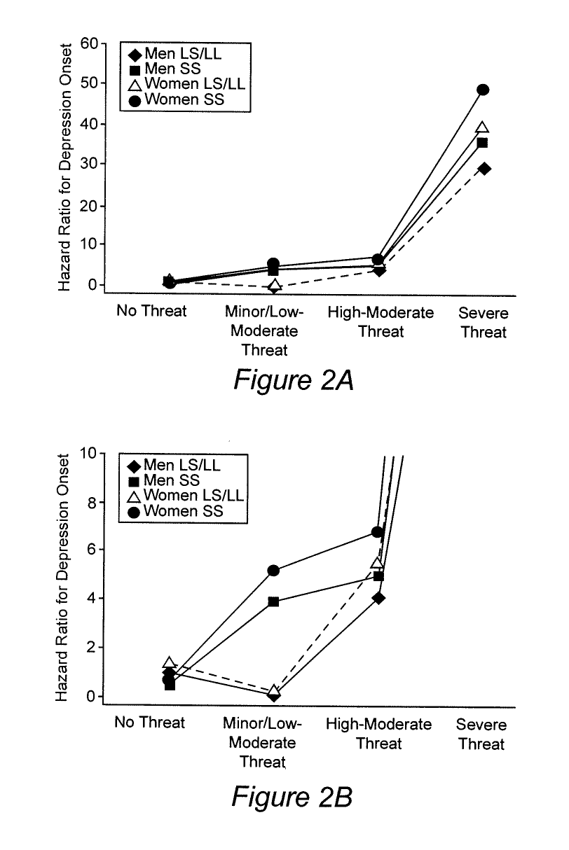

These results, along with 95% CIs, are presented in the table and illustrated in FIG. 2a (the overall results of the best-fit model) and FIG. 2b (which “zooms in” at the critical part of the curve at the mild level of LTCT). Four findings from the best-fit model are noteworthy. First, as seen previously ⁴, the HR for MD increases with higher levels of LTCT with the effect being particularly marked when moving from high moderate to severe levels of LTCT. Second, at every level of threat and genotype, the HR for MD is greater in females than in males. Third, at LTCT levels of 3 and 4, in both males and females, the HR is greater for those with the SS than with the SL or LL genotypes but the difference is small. Fourth, (as most clearly seen in FIG. 2b), at mild and low moderate levels of threat (LTCT=1 in the figure), the differences in risk between those with SS versus SL or LL genotypes is substantial. The risk for a depressive onset is actually decreased for individuals with SL or LL genotypes when they experienced a SLE with a mild level of threat compared to no life event at all. However, for individuals with an SS genotype, the risk for an episode of MD is over 8 times greater in the presence of a mild or low moderate threat event compared to months with no reported SLE.

Prediction of Generalized Anxiety Syndrome

Initially the full model was applied, including levels of LTCT, to predict onsets of GAS. 5-HTT genotype had no effect on risk for GAS either as a main effect or in interaction with levels of LTCT. Reducing the model did not reveal a significant genetic effect. To the prediction of onsets of GAS the final best fit model for MD was applied. The main effects of 5-HTT genotype (χ₁²=0.05, NS), sex (χ₁²=1.22, NS) and LTCT. 4 (χ₁²=0.06, NS) were all nonsignificant. By contrast, the remaining two main effects of levels of stress were both significant: LTCT. 1 (χ₁²=5.41, p=0.02) and LTCT. 3 (χ₁²=10.68, p=0.001). No significant interactions were observed between genotype and LTCT. 1 (χ₁²=0.02, NS) or LTCT. 3 (χ₁²=^{0.75}, NS).

Discussion

Replication of Interaction

Using different measures of SLEs and different analytic methods, a greater degree of temporal resolution was found for concluding that the length-polymorphism in the 5HTT promoter modified the depressogenic effects of SLEs ⁸, compared to the original report by Caspi et al.

The genotype results in this Example differed from Caspi et al. in the following. In the original analyses ⁸, largest differences in stress-responsivity were between those with the LL genotype and those with the SS and SL genotype ⁸. By contrast, in this Example significant differences were found only between those with the SS versus the SL or LL genotype.

To date, association studies for complex human behavioral traits have been problematic, producing low rates of replication. This has arisen from many causes, including low a priori probability, low power, and use of a liberal alpha levels ^{30 31}. These cautions are probably less relevant to the findings of this Example because a prior report (Caspi) was replicated and analyses were performed on a single marker. However, neither the findings of this Example nor those reported by Caspi et al ⁸ are typical association studies. Instead of a main effect of genotype on phenotype, these reports examine genotype-environment interactions. Because interactions are harder to detect than main effects ^{32 33}, replications might be expected to be rarer and hence of particular value when they occur.

The interpretation of genotype-environment interaction can be confounded by genotype-environment correlation. However, this is unlikely to be a concern in this Example because the 5-HTT genotype was found to predict neither exposure to SLEs in general nor specifically to SLEs with high threat levels.

Dose-Response Curve

The impact of the 5-HTT polymorphism on the “dose-response” relationship between stress and risk for MD was clarified as follows in this Example. From the results presented by Caspi et al, the initial expectation was that differences in risk as a function of genotype would grow larger as stress levels increased. Unexpectedly this was not observed. Instead, an increased sensitivity of individuals with the SS genotype to the depressogenic effects of SLEs with mild or low moderate LTCT was observed. The initial evidence in this Example for the interaction between 5HTT genotype and the presence/absence of SLEs was due to this effect, and was so robust because events at these mild levels of threat are more common than events with severe LTCT.

The inventor's finding in this Example—that the genotype-environment interaction is due to a “left-ward” shift in the dose response curve such that SS individuals have increased sensitivity only to mild SLEs—is intriguing. Such a finding suggests that understanding the pathway from genetic variation to clinical disorder in psychiatry may require refined measures of environmental risk factors. Increasing evidence of genetic involvement in the etiology of psychiatric disorders can be interpreted as supportive of the reductionist agenda in psychiatry which seeks to develop etiologic theories for psychiatric disorders in purely molecular terms. The results argue against this as they suggest that understanding gene action in depression requires us to both “go down” to individual genetic polymorphisms and “go out” into the environment with detailed measurements of stressful experiences.

Diagnostic Specificity

In this Example, the specificity of the action of 5-HTT in its modification of the pathogenic effects of SLEs was clarified. Twin studies have suggested a high degree of overlap of genetic risk factors for MD and GAD ^{17 18} and the anxiogenic and depressogenic effects of SLEs are only partially distinct ^{19 37 13}. Therefore, it would be expected that the 5-HTT polymorphism would also modulate the anxiogenic effects of SLEs. However, no such effect was found in this Example. This result suggests some specificity in the modulation of the effects of stress by functional variation in the serotonin transporter.

In this Example, findings were based on twins from one racial and geographical region. Analyses assumed that when SLEs occurred in the same month as depressive onsets, the SLE preceded the onset. In two prior studies, additional interview material was used to determine, when SLEs and depressive onsets co-occurred in the same month, that in nearly all instances, the SLE preceded rather than followed the onset ^{5 13}.

Overall Significance

A recent meta-analysis of the 5-HTT polymorphism and MD, including 11 studies with 941 patients and 2110 controls, concluded that the studies were homogeneous and the association was not significant (with an pooled OR and 95% CIs of 1.08 and 0.96-1.22 ³⁸). Another recent meta-analysis examined the association between this polymorphism and “avoidance-related” personality traits, which includes neuroticism and related constructs which have been shown in both genetic and prospective designs to be strongly related to risk for MD ^{39 40 41 42}. Their analyses of 22 studies suggest a quite modest relationship with a mean difference of 0.11 SD units, (95% CI 0.06-0.17) ⁴³. These results suggest that the straight-forward association between variation in the 44 bp insertion deletion polymorphism in the 5-HTT gene and risk for the clinical syndrome of MD or associated personality traits is subtle at best. The results of this Example suggest that the 5HTT may be an example of a gene that influences liability to MD not by a main effect on risk, but rather by control of sensitivity to the pathogenic effects of the environment ⁴⁴.

Thus, the present inventor has discovered that variants in the human serotonin transporter gene impact on the pattern of an individual's response to minor to low moderately threatening stressful life events. Both men and women who possess two copies of the “short” allele have been found to be much more sensitive to the depressogenic effects of mildly threatening events than are those who have either a long-short or a long-long genotype.

While the invention has been described in terms of its preferred embodiments, those skilled in the art will recognize that the invention can be practiced with modification within the spirit and scope of the appended claims.

### REFERENCES

1. Kessler R C. The effects of stressful life events on depression. Ann Rev Psychology. 1997; 48:191-214.
2. Tennant C. Life events, stress and depression: a review of recent findings. Aust N Z J Psychiatry. 2002; 36:173-182.
3. Kendler K S, Karkowski L M, Prescott C A. Causal relationship between stressful life events and the onset of major depression. Am J Psychiatry. 1999; 156:837-841.
4. Kendler K S, Kuhn J, Prescott C A. The interrelationship of neuroticism, sex, and stressful life events in the prediction of episodes of major depression. Am J Psychiatry. 2004; 161:631-636.
5. Kendler K S, Kessler R C, Walters E E, MacLean C J, Sham P C, Neale M C, Heath A C, Eaves L J. Stressful life events, genetic liability and onset of an episode of major depression in women. Am J Psychiatry. 1995; 152:833-842.
6. Silberg J, Rutter M, Neale M, Eaves L. Genetic moderation of environmental risk for depression and anxiety in adolescent girls. Br J Psychiatry. 2001; 179:116-121.
7. Eaves L, Silberg J, Erkanli A. Resolving multiple epigenetic pathways to adolescent depression. Journal of Child Psychology and Psychiatry and Allied Disciplines. 2003; 44:1006-1014.
8. Caspi A, Sugden K, Moffitt T E, Taylor A, Craig I W, Harrington H, McClay J, Mill J, Martin J, Braithwaite A, Poulton R. Influence of life stress on depression: moderation by a polymorphism in the 5-HTT gene. Science. 2003; 301:386-389.
9. Costello C G. Social factors associated with depression: a retrospective community study. Psychol Med. 1982; 12:329-339.
10. Brugha T S, Bebbington P, Sturt E, MacCarthy B, Wykes T. The relation between life events and social support networks in a clinically depressed cohort. Soc Psychiatry Psychiatr Epidemiol. 1990; 25:308-313.
11. Brown G W, Harris T O. Social Origins of Depression: A Study of Psychiatric Disorder in Women. London: Tavistock; 1978.
12. Bland R C, Om H, Newman S C. Lifetime prevalence of psychiatric disorders in Edmonton. Acta Psychiatr Scand. 1988; 77 (suppl 338):24-32.
13. Kendler K S, Karkowski L, Prescott C A. Stressful life events and major depression: risk period, long-term contextual threat and diagnostic specificity. J Nerv Ment Dis. 1998; 186:661-669.
14. Thoits P A. Dimensions of life events that influence psychological distress: An evaluation and synthesis of the literature. In: Kaplan H B, ed. Psychosocial Stress: Trends in Theory and Research. New York: Academic Press; 1983:33-102.
15. Brown G W, Sklair F, Harris T O, Birley J L T. Life events and psychiatric disorders. I. Some methodological issues. Psychol Med. 1973; 3:74-87.
16. Paykel E S. Stress and affective disorders in humans. Semin Clin Neuropsychiatry. 2001; 6:4-11.
17. Kendler K S, Neale M C, Kessler R C, Heath A C, Eaves L J. Major depression and generalized anxiety disorder: same genes, (partly) different environments? Arch Gen Psychiatry. 1992; 49:716-722.
18. Roy M-A, Neale M C, Pedersen N L, Mathe A A, Kendler K S. A twin study of generalized anxiety disorder and major depression. Psychol Med. 1995; 25:1037-1049.
19. Finlay-Jones R, Brown G W. Types of stressful life events and the onset of anxiety and depressive disorders. Psychol Med. 1981; 11:803-815.
20. Kendler K S, Prescott C A. A population-based twin study of lifetime major depression in men and women. Arch Gen Psychiatry. 1999; 56:39-44.
21. American Psychiatric Association. Diagnostic and Statistical Manual of Mental Disorders, Revised Third Edition. Washington, D.C.: American Psychiatric Association; 1987.
22. Cohen J. A coefficient of agreement for nominal scales. Educ Psychol Meas. 1960; 20:37-46.
23. Kendler K S, Hettema J M, Butera F, Gardner C O, Prescott C A. Life event dimensions of loss, humiliation, entrapment, and danger in the prediction of onsets of major depression and generalized anxiety. Arch Gen Psychiatry. 2003; 60:789-796.
24. Brown G W. Life events and measurement. In: Brown G W, Harris T O, eds. Life Events and Illness. New York: The Guilford Press; 1989:3-45.
25. Fleiss J S, Cohen J, Everett B S. Large sample standard errors of kappa and weighted kappa. Psychol Bull. 1969; 72:323-327.
26. Stoltenberg S F, Twitchell G R, Hanna G L, Cook E H, Fitzgerald H E, Zucker R A, Little K Y. Serotonin transporter promoter polymorphism, peripheral indexes of serotonin function, and personality measures in families with alcoholism. Am J Med Genet. 2002; 114:230-234.
27. Survival Analysis Using The SAS System: A Practical Guide. 2nd ed. Cary, N.C.: SAS Institute Inc.; 1995:-292.
28. Cox D R. Regression models and life tables (with discussion). J Roy Stat Soc, Ser B. 1972; 34:187-220.
29. Masters T. Practical Neural Network Recipes in C++. Boston, Mass.: Academic Press; 1993.
30. Sullivan P F, Eaves L J, Kendler K S, Neale M C. Genetic case-control association studies in neuropsychiatry. Arch Gen Psychiatry. 2001; 58:1015-1024.
31. Lohmueller K E, Pearce C L, Pike M, Lander E S, Hirschhorn J N. Meta-analysis of genetic association studies supports a contribution of common variants to susceptibility to common disease. Nat Genet. 2003; 33:177-182.
32. McCall R B. So many interactions, so little evidence. Why? In: Wachs T D, Plomin R, eds. Conceptualization and Measurement of Organism-Environment Interaction. Washington, D.C.: American Psychological Association; 1991:142-161.
33. Wahlsten D. Insensitivity of the analysis of variance to heredity-environment interaction. Behav Brain Sci. 1990; 13:109-161.
34. Gillespie, N A, Whitfield, J. B., Williams, D, Heath, A. C., and Martin, N. G. The relationship between stressful life events, the serotonin transporter (5-HTTLPR) genotype and major depression. Psychological Medicine. 2004. Cambridge, UK, Cambridge University Press.
35. Eley T C, Sugden K, Corsico A, Gregory A M, Sham P, McGuffin P, Plomin R, Craig I W. Gene-environment interaction analysis of serotonin system markers with adolescent depression. Mol Psychiatry. 2004.
36. Grabe H J, Lange M, Wolff B, Volzke H, Lucht M, Freyberger H J, John U, Cascorbi I. Mental and physical distress is modulated by a polymorphism in the 5-HT transporter gene interacting with social stressors and chronic disease burden. Mol Psychiatry. 2004.
37. Barrett J E. The relationship of life events to the onset of neurotic disorders. In: Barrett J E, Rose R M, Klerman G L, eds. Stress and Mental Disorder. New York: Raven Press; 1979:87-109.
38. Anguelova M, Benkelfat C, Turecki G. A systematic review of association studies investigating genes coding for serotonin receptors and the serotonin transporter: I. Affective disorders. Mol Psychiatry. 2003; 8:574-591.
39. Angst J, Clayton P. Premorbid personality of depressive, bipolar, and schizophrenic patients with special reference to suicidal issues. Compr Psychiatry. 1986; 27:511-532.
40. Hirschfeld R M A, Klerman G L, Lavori P W, Keller M B, Griffith P, Coryell W. Premorbid personality assessments of first onset of major depression. Arch Gen Psychiatry. 1989; 46:345-350.
41. Kendler K S, Neale M C, Kessler R C, Heath A C, Eaves L J. A longitudinal twin study of personality and major depression in women. Arch Gen Psychiatry. 1993; 50:853-862.
42. Fanous A, Gardner C O, Prescott C A, Cancro R, Kendler K S. Neuroticism, major depression and gender: a population-based twin study. Psychol Med. 2002; 32:719-728.
43. Munafo M R, Clark T G, Moore L R, Payne E, Walton R, Flint J. Genetic polymorphisms and personality in healthy adults: a systematic review and meta-analysis. Mol Psychiatry. 2003; 8:471-484.
44. Kendler K S, Eaves L J. Models for the joint effect of genotype and environment on liability to psychiatric illness. Am J Psychiatry. 1986; 143:279-289.

1. A method of identifying an individual that is at risk for an episode of major depression, comprising the steps of
identifying an individual that possesses an SS allelic variant of human serotonin transporter gene;
providing a scale which correlates life events experienced by human beings with a level of severity, wherein at least some of said life events on said scale are deemed to be of mild or low moderate long term contextual threat; and
determining whether, for said individual, a life event which has a severity of mild or greater on said scale has or is likely or certain to occur; and, if said life event has or is likely or certain to occur,
concluding that said individual is at risk for an episode of major depression.

2. The method of claim 1, wherein said life event has a severity of mild or low moderate on said scale.

3. A method for treating an individual that is either at risk or is experiencing an episode of major depression, comprising the steps of
identifying an individual that possesses an SS allelic variant of human serotonin transporter gene;
providing a scale which correlates life events experienced by human beings with a level of severity, wherein at least some of said life events on said scale are deemed to be mild or low moderate long term contextual threat; and
determining whether, for said individual, a life event which has a severity of mild or greater on said scale has or is likely or certain to occur; and, if said life event has or is likely or certain to occur,
providing said individual with a treatment which alleviates or prevents an episode of major depression.

4. The method of claim 3, wherein said treatment includes counseling.

5. The method of claim 3, wherein said treatment includes pharmaceuticals.

6. The method of claim 1, including presymptomatic identification of an at risk individual.

7. The method of claim 3, including presymptomatic identification of an at risk individual.

8-9. (canceled)

10. A method of identifying an individual that is at risk for an episode of major depression, comprising the steps of
identifying an individual that possesses an SS allelic variant of human serotonin transporter gene;
determining whether, for said individual, a stressful life event has or is likely or certain to occur;
assessing whether said stressful life event is of mild or low moderate long term contextual threat; and, if said life event has or is likely or certain to occur, and if said life event is of mild or low moderate long term contextual threat,
concluding that said individual is at risk for an episode of major depression.

11. A method for treating an individual that is either at risk or is experiencing an episode of major depression, comprising the steps of
identifying an individual that possesses an SS allelic variant of human serotonin transporter gene;
determining whether, for said individual, a stressful life event has or is likely or certain to occur;
assessing whether said stressful life event is of mild or low moderate long term contextual threat; and, if said life event has or is likely or certain to occur, and if said life event is of mild or low moderate long term contextual threat,
concluding that said individual is at risk for an episode of major depression; and,
providing said individual with a treatment which alleviates or prevents said episode of major depression.

12. The method of claim 11, wherein said treatment includes counseling.

13. The method of claim 11, wherein said treatment includes pharmaceuticals.

14. The method of claim 11, including presymptomatic identification of an at risk individual.
